(19) United States
(12) Patent Application Publication
(10) Pub. No.: US 2009/0093744 A1
(43) Pub. Date: Apr. 9, 2009

(54) Brace for Reducing a Metacarpal Fracture

(76) Inventor: Robert MacArthur, Irvine, CA (US)

Correspondence Address: FISH & ASSOCIATES, PC;ROBERT D. FISH, 2603 Main Street, Suite 1050, Irvine, CA, 92614-6232, US

(21) Appl. No.: 12/245,826

(22) Filed: Oct. 6, 2008

Related U.S. Application Data
(60) Provisional application No. 60/998,311, filed on Oct. 9, 2007.

(51) Int. Cl. A61F 5/00

(52) U.S. Cl. 602/22; 602/21

(57) ABSTRACT

A brace for reducing a metacarpal fracture is presented. The brace comprises a palm plate and a finger trough for receiving a finger having a metacarpal fracture. The trough couples to the palm plate at a proper angle of flexion for reducing the fracture. An individual places the brace on the plamar side of their hand and places the finger having the fracture in the trough. A fastening system holds the brace in a stable position on the hand. The trough maintains the metacarpal-phalangeal joint at the proper angle of flexion while also maintaining necessary axial compression along the finger to reduce the fracture.

This application claims the benefit of priority to U.S. provisional application having Ser. No. 60/998,311 filed on Oct. 7, 2007. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is fracture reduction technologies.

### BACKGROUND

Metacarpal fractures, a boxer's fracture for example, are difficult to correct because the metacarpal-phalangeal (MP) joint should be immobilized at a proper flexion angle (e.g., about 90 degrees) to promote proper healing. The standard reduction technique for a boxer's fracture includes flexing the little finger's MP joint to 90 degrees and pushing axially along the finger to place the fracture in a proper position for healing. Once in position, the MP joint and finger should remain immobilized until healing is complete after six to twelve weeks.

Various braces for immobilizing the MP joint and finger in response to a metacarpal facture are commonly available. One example is U.S. Pat. No. 4,294,237 to Frazier titled “Splint for Reducing Fractures of the Metacarpals” filed on Dec. 26, 1979, which describes using a pressure plate to reduce the apex of a broken metacarpal. Another example includes U.S. Pat. No. 4,366,812 to Nuzzo titled “Adjustable Digital and Metacarpal Splint Apparatus” filed on May 26, 1981, which discloses the use of an elongated splint having an adjustable position. Yet another example is U.S. Pat. No. 4,662,364 to Viegas et al. titled “Method and Brace to Immobilize Fractures” filed on Apr. 2, 1985, which discusses using pads on an adjustable brace to apply pressure in a desired area of a facture. And yet another example includes U.S. Pat. No. 4,813,406 to Ogle titled “Orthopedic Splint Arrangement” filed on Aug. 6, 1986, which describes using a ring-like splint to immobilize a portion of the body, including a digital member. These and other braces fail to achieve proper fracture reduction due to the hypothenar pad and required pressure on the palmar side of the MP joint, thereby immobilizing the MP joint in extension. Keeping the MP joint in extension can cause permanent extension contracture. Although the above braces have found their respective niches in the market, they collectively fail to offer the standard reduction maneuver for a metacarpal fracture over an extended period of time and, among other issues, fail to allow for mobility of remaining fingers.

Ideally a brace for reducing a metacarpal fracture would immobilize the MP joint and finger at a proper flexion angle for extended periods of time while also allowing the remaining fingers on the hand to have a high degree of mobility. It has yet to be appreciated that a brace can be constructed where the MP joint is held at proper flexion angle while also applying an axial pressure along the finger to reduce the fracture.

Thus, there is still a need for braces that reduce metacarpal fractions.

### SUMMARY OF THE INVENTION

The present invention provides apparatus, systems and methods in which a metacarpal fracture (e.g., a boxer's fracture) can be secured using a brace to reduce the fracture. The brace includes a palm plate and a finger trough coupled to the palm plate at a proper angle of flexion. The brace can also include a support tube that can be placed around the finger having the fracture. The tube and finger can be inserted into the trough. An adjuster coupled to the trough can position the tube by moving the tube axially toward the head of the fractured metacarpal causing the finger to apply appropriate axial compression to the fractured metacarpal, restoring it to a proper position. The brace also preferably includes a fastener system that holds the palm plate and trough in a stable position on an individual's hand. In a preferred embodiment, the palm plate, trough, and support tube are substantially radiolucent.

In one aspect of the inventive subject matter, the brace can include an additional trough for immobilizing a finger adjacent to the finger having the fractured metacarpal. In some embodiments the additional trough is removeably affixed to the palm plate, and possibly to the first finger trough, to support manufacturing a single brace design that can operate on either the left or right hand.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1 is a schematic of a hand having a metacarpal fracture.

FIG. 2A is a schematic of a brace having a finger trough coupled to a palm plate at a proper flexion angle with respect to the palm plate.

FIG. 2B is a schematic of a brace having multiple finger troughs.

FIG. 3 is schematic of a brace having support tube disposed within a finger trough.

FIG. 4 is a schematic of fracture reduced by the contemplated brace.

FIG. 5 is a schematic of a hand wearing a brace strapped to a hand via a fastening system.

### DETAILED DESCRIPTION

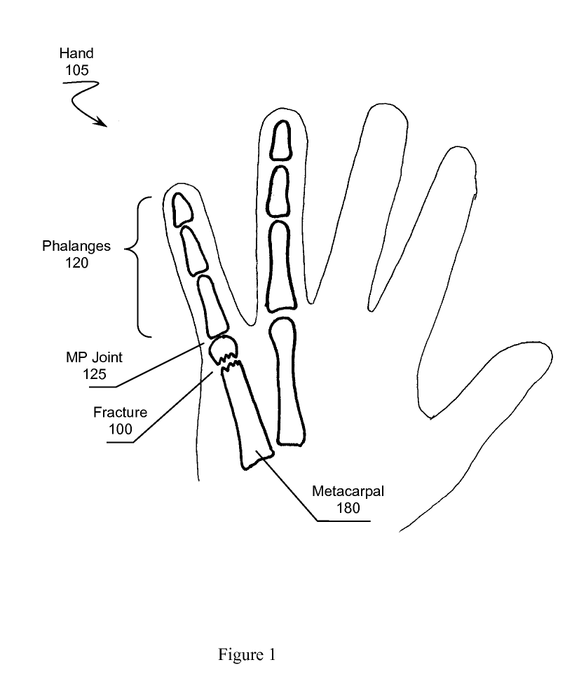

In FIG. 1, hand 105 has experienced a metacarpal fracture 100, for example a boxer's fracture. Fracture 100 can be reduced by the use of a brace that induces a proper angle of flexion (e.g., about a 90 degree flexion angle) at MP joint 125 located between phalanges 120 and metacarpal 180. The brace can also be constructed to apply an axial pressure along phalanges 120 to reduce the apex of fracture 100.

### Brace Overview

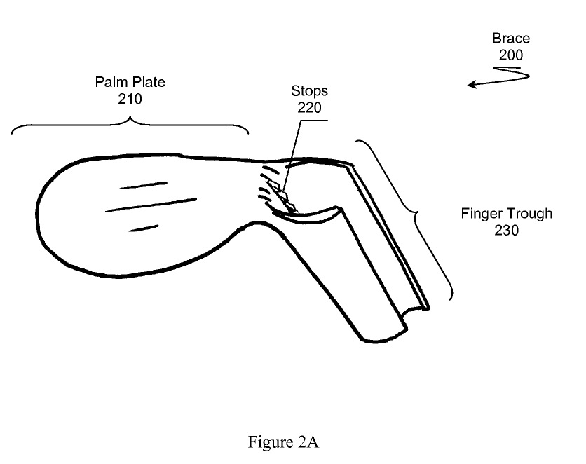

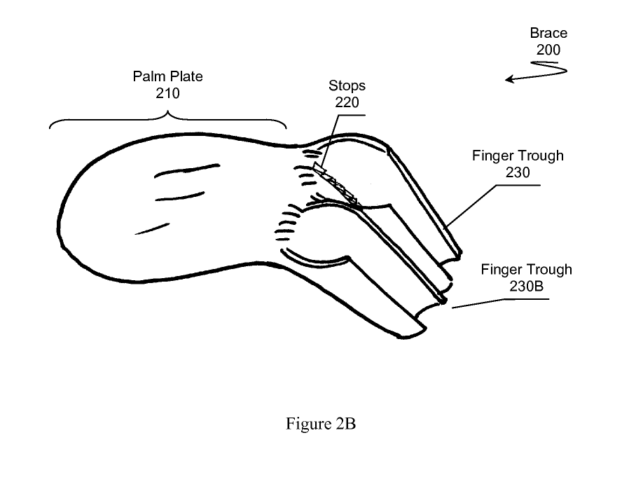

In FIG. 2A, brace 200 comprises palm plate 210 and finger trough 230 that provide for proper reduction of a metacarpal fracture. Palm plate 210 preferably is placed on the palmar side of a hand having a metacarpal fracture. The finger associated with the metacarpal fracture is inserted into trough 230. Finger trough 230 and plate 210 cause the finger to reside at a proper angle of flexion.

Palm plate 210 approximately defines a plane that is roughly parallel to the metacarpals of the hand having the fracture. The angle of flexion provided by finger trough 230, as discussed below, is described with respect to the plane of plate 210 for ease of description. However, one skilled in the art will recognize that angle of flexion provided trough 230 also corresponds to a proper angle of flexion at the MP joint of the fracture.

Although plate 210 is illustrated as flat, it should be noted that palm plate 210 can be also be contoured to fit a human hand. It is also contemplated that palm plate 210 can be manufactured out of a moldable material to allow for custom fitting plate 210 to the hand having a fracture. An example of an acceptable material includes KYDEX® Thermoplastic Sheets available from Kleerdex Company, LLC.

Finger trough 230 is preferably coupled to palm plate 210 to form a proper flexion angle for a fracture. As used herein “a proper flexion angle” should be broadly interpreted to include angles that provide sufficient flexion for adequate healing of a metacarpal fracture. In a preferred embodiment, a proper flexion angle includes flexion angles between about 70 degrees to about 100 degrees, inclusively. In a more preferred embodiment to address a boxer's fracture, a proper flexion angle is between about 85 degrees to about 95 degrees, inclusively. In yet more preferred embodiments, the proper flexion angle is about 90 degrees. One skilled in the art will recognize that other angles are also possible depending on the nature or location of the fracture, all of which are contemplated.

One should note the value of having a brace that maintains the proper angle of flexion at the MP joint. When the MP joint is in flexion during healing, the collateral ligaments on either side of the MP joint remain flexed thereby ensuring the finger and hand retains flexibility after the fracture has healed. Otherwise, if the MP joint is in extension during healing, the collateral ligaments could tighten causing loss of mobility of the finger or permanent contracture.

In some embodiments, trough 230 and plate 210 are molded as a single work piece. For example, brace 200 could be formed through an injection mold process. It other more modular embodiments, trough 230 could be a separate component that couples to the plate 210 mechanically (e.g., slot and tabs, bolts, snaps, etc . . . ) or chemically (e.g., glue, thermal, etc . . . ) to form a rigid bracing structure. It some embodiments, trough 230 can have its flexion angle adjusted as desired for a patient. For example, in embodiments where brace 200 is a single work piece and is formed from a thermoplastic, brace 200 can be heated and trough 230 can be adjusted to a proper angle of flexion. In modular embodiments, trough 230 can be attached to plate 210 at a desirable angle.

In a preferred embodiment, trough 230 is configured to restrict or otherwise immobilize a finger disposed within the trough. Finger movement can be restricted using any suitable method. In some embodiments, finger trough 230 can be tapered to hold the finger tightly. Additionally, trough 230 can be formed with an overly small diameter and having an open face as shown in FIG. 2A. When a finger is inserted into such a trough, the sides of trough 230 expand to receive the finger while also splinting the finger. In yet another embodiment, the finger can be wrapped in padding, possibly initially disposed within trough 230, to provide a snug fit within trough 230.

Trough 230 also preferably applies axial pressure (e.g., force along the finger toward the MP joint) to a finger. For example, in embodiments where trough 230 it tapered, a finger within the trough is forced back toward the MP joint in a reduction maneuver where the finger is pushed against the metacarpal head to reduce the fracture. The same axial pressure can also be achieved through the use of padding.

In yet another embodiment, brace 200 can include an additional support tube that can be disposed within trough 230. The support tube can also be used to restrict movement of a finger and apply axial pressure. For example, the position of the support tube can be adjusted by positioning the tube at different points along stops 220. Support tubes are discussed in greater detail below.

One skilled in the art will appreciate that trough 230 can take on many different forms while still falling within the scope of the inventive subject matter. For example, trough 230 can be a full, hollow cylinder that substantially covers a received finger, or trough 230 can have one or more open faces as shown. It is also contemplated that trough 230 can include one or more vents or other holes to allow free flow of air in and around a finger disposed in trough 230.

In a preferred embodiment, brace 200 having plate 210 and trough 230 are manufactured to fit either the left or right hand. Brace 200 can be customized at the time of use to provide a proper fit. For example, when brace 200 comprises a thermoplastic, brace 200 can be heated and then molded to fit comfortably on an individual's hand. Once cooled, brace 200 will again be rigid and provide the necessary support or reduction.

FIG. 2B presents an additional exemplary embodiment of brace 200 having multiple finger troughs 230 and 230B. Finger trough 230B also provides further movement restriction by restricting the movement of adjacent fingers to the finger having a fracture. For example, a little finger can be placed within trough 230 while a ring finger is placed in trough 230B.

Finger trough 230B also preferably couples to palm plate 210 in a similar fashion as trough 230; molded as a single unit, mechanically coupled, or chemically coupled. In some embodiments, trough 230B is a separate component that can be coupled on either side of trough 230. For example, trough 230B can be removeably affixed to palm plate allowing for replacement or for adjusting placement (e.g., location or flexion angle) of trough 230B. Such an approach also provide for creating a brace that can be used on either hand.

Trough 230B can also couple to trough 230 to prevent relative movement of fingers disposed within the troughs. The troughs can be coupled mechanically (e.g., straps, snaps, screws, etc . . . ), chemically (e.g., glue, thermal setting, etc . . . ), or other coupling method.

Toughs 230 and 230B preferably can be formed having a “U” shape or “C” shape cross section where the open face allows for easy viewing of fingers disposed within the troughs. As a practitioner fits an individual with brace 200, the practitioner can determine if the fingers and hand have good capillary refill by visually inspecting the finger nail beds through the open faces of the trough.

### Support Tube

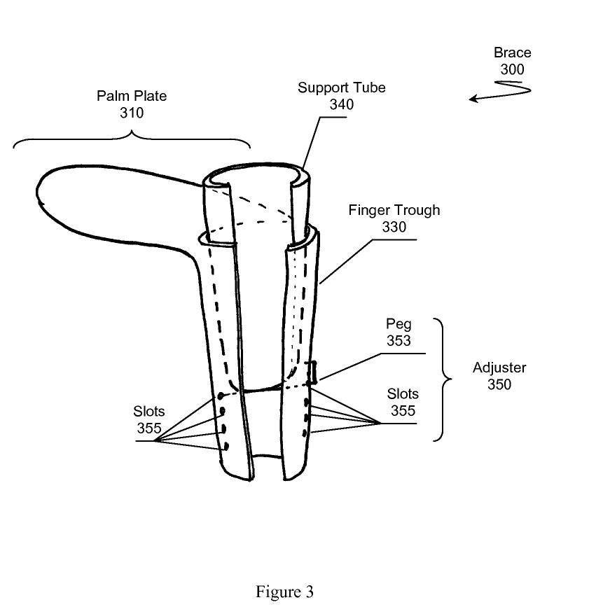

In FIG. 3, brace 300 comprises palm plate 310, trough 330, and an additional, optional support tube 340 that can be inserted within finger trough 330. Support tube 340 is configured to be disposed within trough 330 and to slide axially within the trough toward palm plate 310 to apply sufficient axial pressure to reduce a metacarpal fracture. Brace 300 can apply axial pressure to a finger by positioning tube 340 within trough 330 to a desired position. Adjuster 350 can be used to lock tube 340 in a substantially fixed position to maintain axial compression.

Support tube 340 can comprise a rigid material that can be placed around a finger having a metacarpal fracture and can function as a finger splint. As describe previously with respect to finger troughs, tube 340 can also take on many different forms to restrict movement of the finger. For example, tube 340 can be tapered or made overly small to tightly wrap around the finger. Additionally, padding can be used in conjunction with tube 340 to immobilize the finger. It is also contemplated that tube 340 can comprise flexible material or could even, in fact, be the padding itself. Regardless of the material used to construct tube 340, preferably tube 340 provides for sliding within trough 330 to a desired position to apply proper axial pressure.

In some embodiments, tube 340 couples to trough 330 with a preferred orientation to provide for proper reduction. For example, tube 340 can include rails that slide within tracks placed within trough 340, or vice versa. The rails and tracks can be configured to only support a single configuration of tube 340 and trough 330.

In a preferred embodiment, support tube 340 is adjustable to receive fingers of different dimensions. Providing a “one size fits all” tube 340 reduces manufacturing costs while also ensuring that tube 340 offers necessary support. For example, tube 340 could be produced with an overly small diameter, having flexible walls, and an open face. Such a tube expands to accommodate an inserted finger. Alternatively, tube 340 can made to have a larger diameter and can include one or more tightening elements (e.g., straps, clasps, snaps, etc . . . ) to adjust or to secure tube 340 to the finger. Furthermore, tube 340 can be made overly long where excess material can be removed from the end of tube 340 to shorten its length to accommodate comfortably shorter fingers or smaller hands.

Embodiments employing tube 340 preferably include tube adjuster 350 coupled to tube 340 and trough 330. Adjuster 350 is configured to position support tube 340 at a substantially fixed position within trough 330 to ensure that tube 340 maintains a proper axial compression or angle of flexion during healing. As shown in FIG. 3, adjuster 350 can include peg 353 and one or more pairs of slots 355. Peg 353 can be inserted into slots 355 to prevent tube 340 from sliding out of position where the counter force of the finger causes tube 340 to rest against peg 353. It is also contemplated that tube 340 could include slots through which peg 353 could penetrate to hold tube 340 in a fixed position relative to trough 330.

It should be noted that other forms of adjuster 350 can be used while still falling within the scope of the inventive subject matter. For example, tube 340 and trough 330 can couple to each other via a rack and pinion assembly. The rack can be placed on a surface of tube 340 or on a surface of trough 340 (see stops 220 of FIGS. 2A and 2B). As the pinion of the adjuster is rotated, tube 340 can travel along the rack to a desirable position that provides proper axial compression. Use of a rack and pinion assembly provides for fine grained adjustment of the axial pressure supplied by tube 340 to reduce the metacarpal fracture.

Preferably adjuster 350, regardless of its form, includes a lock that locks support tube 340 at a substantially fixed position. The lock can simply prevent slipping in a distal direction as shown by peg 353. Alternatively the lock can prevent any relative movement of tube 340 within trough 330. As shown, peg 353 is locked into positing by slots 355 and via a hook that latches into a slot to prevent peg 353 from sliding out or becoming lost. Additionally, peg 353 can pass through tube 340 to prevent any relative movement between tube 340 and trough 330. Another example of a lock useful in embodiments having a rack and pinion adjuster includes using a pawl to lock the pinion and prevent tube 340 from sliding freely within trough 330.

### Reduction of Metacarpal Fracture

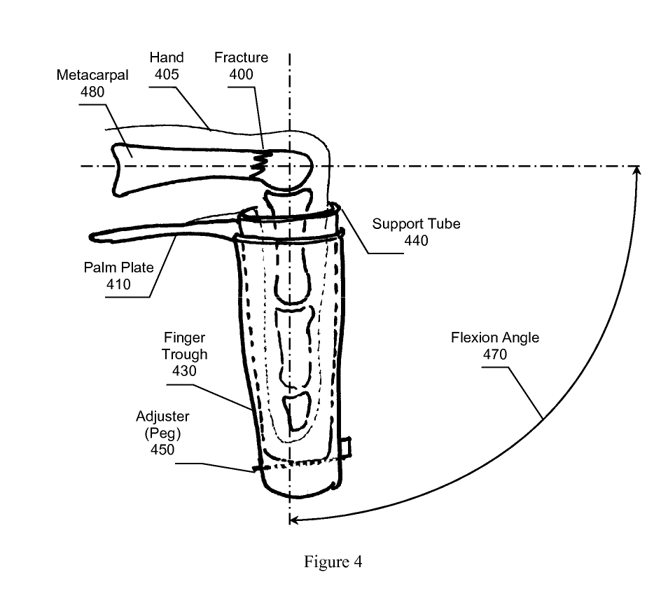

FIG. 4 illustrates reduction of metacarpal fracture 400 on hand 405 through the use of a brace comprising palm plate 410, trough 430, and support tube 440. The finger having fracture 400 is placed within support tube 440 which in turn is placed within trough 430. Axial pressure is applied to the finger by adjusting the position of tube 440 using adjuster 450. The rigid support provided by palm plate 410 and trough 430 ensures that the MP joint maintains proper flexion angle 470 relative to metacarpal 480. Tube 440 also maintains proper axial compression while the brace is worn.

Although FIG. 4 illustrates the use of a support tube 440, it should be noted that tube 440 could be omitted from the brace. For example, the finger could be disposed directly within trough 430 without additional support. In some embodiments lacking support tube 440, the finger disposed within trough 430 is wrapped or at least partially covered by padding (e.g., foam). It is also contemplated the padding can be disposed on the inner surface of trough 430 as previously described.

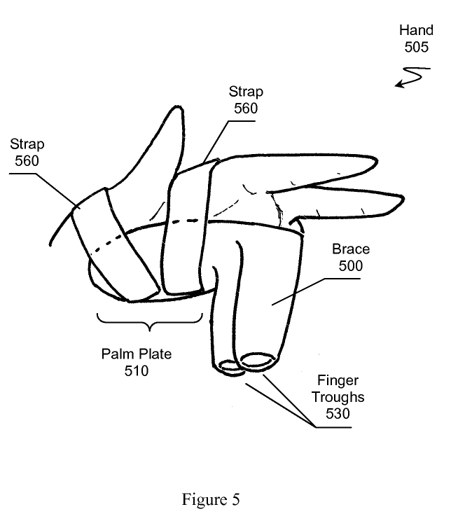

In FIG. 5, hand 505 is wearing a brace 500 that is configured according to the disclosed inventive subject matter. In the example shown, the little finger of hand 505 has experienced a boxer's fracture and is properly reduced by brace 500. The little finger and ring finger of hand 505 are placed within troughs 530 to restrict or otherwise immobilize the fingers. Troughs 530 are coupled to palm plate 510 and together and form a proper flexion angle to promote healing. Brace 500 is secured to hand 505 via a fastening system that holds plate 510 and trough 530 in a stable position to maintain a proper standard reduction position for an extended period of time. The fastening system preferably also allows an individual to remove the brace when desirable for various activities including cleaning the hand, showering, or other circumstances.

A brace fastening system preferably binds brace 500 to hand 505 without substantially restricting movement of the thumb, index finger, or middle finger. In some embodiments having a single trough for the little finger, the ring finger is also free to move. In a preferred embodiment, the fastening system includes a plurality of straps 560 that can wrap around the back of the hand, around the thumb, and attach to palm plate 510. Suitable material for straps 560 comprise hook and loop fasteners including Velcro® brand fasteners

One skilled the art will appreciate that brace 500 can include numerous variations while still falling within the scope of the inventive subject matter. For example, brace 500 can be made from various materials to provide advantageous characteristics, can comprise modular components, or can comprise adjustable parts.

Brace 500 preferably comprises a rigid material that can support hand 505 during healing. Preferred rigid materials also offer the ability to customize brace 500 to the shape or contours of hand 505. For example, brace 500 can be comprised of thermoplastic that can be heated and molded to a person's hand. In addition, preferred materials can include radiolucent materials that allow a practitioner to adjust brace 500 for proper reduction while viewing a fracture via a radiographic image or through live fluoroscopy.

Although in a preferred embodiment plate 510 and one or more of troughs 530 are formed as a single work piece, it is also contemplated that Brace 500 can comprise modular components. For example plate 510, a support tube, and trough 530 can be produced separately according to various sizes and shapes. The individual components can be combined or locked together in desirable positions or angles to form brace 500. A module brace system allows for addressing a wide range of hand or finger dimensions as well as for addressing extreme scenarios where a “one size fits all” brace would not be practical.

It is also contemplated that the components of brace 500 can be individually adjustable. In some embodiments, the various components (e.g., plate 510, troughs 530, a support tube, etc . . . ) are manufactured as being overly large including excess material. Excess material can be removed so that brace 500 properly fits a specific hand. In other embodiments, the components can include tightening elements to ensure snug fits as previously discussed.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A brace for reducing a metacarpal fracture, comprising:
a palm plate;
a first finger trough coupled to the palm plate at a proper flexion angle with respect to a plane defined approximately by the palm plate where the trough restricts movement of a finger disposed within the trough and applies axial pressure to the finger toward a metacarpal head to reduce a metacarpal fracture of the finger; and
a fastener system adapted to hold the palm plate and the first finger trough in a stable position on an individual's hand having the fracture.

2. The brace of claim 1, further comprising a support tube capable of receiving the finger, and that can (a) be disposed within the first finger trough and (b) slide axially within the first finger trough toward the metacarpal head to reduce the metacarpal fracture of the finger.

4. The brace of claim 2, wherein the support tube is adjustable to receive fingers of different dimensions.

3. The brace of claim 2, further comprise an adjuster coupled to the first finger trough and the support tube, and configured to position the support tube at a substantially fixed position within the first finger trough.

5. The brace of claim 3, wherein the adjuster comprises a rack and a pinion.

6. The brace of claim 3, wherein the adjuster comprises a slot and a peg.

7. The brace of claim 3, wherein the adjuster comprises a lock that locks the support tube in the fixed position.

8. The brace of claim 1, further comprising a second finger trough coupled to the palm plate at the proper flexion angle with respect to the plane defined approximately by the palm plate.

9. The brace of claim 8, wherein the first finger trough is configured to restrict movement of a little finger of the hand when the little finger is disposed within the first finger trough.

10. The brace of claim 8, wherein the second finger trough is configured to restrict movement of a ring finger of the hand.

11. The brace of claim 8, wherein the second finger trough is removeably affixed to the palm plate.

12. The brace of claim 8, wherein the first and the second finger trough are coupled to each other to prevent relative movement of fingers disposed within the troughs.

13. The brace of claim 1, wherein the fastener system includes a hook and loop fastener.

14. The brace of claim 13, wherein the fastener system includes a plurality of straps.

15. The brace of claim 1, wherein the palm plate and the first finger trough are formed as a single work piece.

16. The brace of claim 1, wherein the palm plate and first finger trough form an assembly that can be used on left and right hands.

17. The brace of claim 1, wherein the palm plate and the first finger trough are formed of material that is substantially radiolucent.

18. The brace of claim 1, further comprising padding located within the first finger trough.

19. The brace of claim 1, wherein the proper angle of flexion is between 70 degrees and 100 degrees, inclusively.

20. The brace of claim 19, wherein the proper angle of flexion is between 85 degrees and 95 degrees, inclusively.
